(19) United States
(12) Patent Application Publication
(10) Pub. No.: US 2017/0188883 A1
(43) Pub. Date: Jul. 6, 2017

(54) POSITION DETECTION SYSTEM AND GUIDANCE SYSTEM

(71) Applicant: OLYMPUS CORPORATION, Tokyo (JP)

(72) Inventor: Atsushi CHIBA, Tokyo (JP)

(73) Assignee: OLYMPUS CORPORATION, Tokyo (JP)

(21) Appl. No.: 15/463,520

(22) Filed: Mar. 20, 2017

Related U.S. Application Data
(63) Continuation of application No. PCT/JP2015/079868, filed on Oct. 22, 2015.

(30) Foreign Application Priority Data
Mar. 25, 2015 (JP) 2015-063108

(51) Int. Cl. A61B 5/06; A61B 1/00; A61B 1/04

(52) U.S. Cl. CPC A61B 5/062; A61B 1/041; A61B 1/00158

(57) ABSTRACT

A position detection system includes: a capsule medical device having therein a magnetic field generator for generating a magnetic field; magnetic field detectors for detecting the magnetic field and outputting detection signals; and a processor including hardware. The processor is configured to: calculate a position of the capsule medical device using at least one of the detection signals; determine whether or not proper position detection of the capsule medical device based on the detection signals is possible using a threshold that is a value based on at least one of output values of the detection signals output by the magnetic field detectors under a predetermined condition; decide a determination value using at least one of the output values of the detection signals; and determine that the proper position detection of the capsule medical device is not possible when the determination value is smaller than the threshold.

### CROSS-REFERENCE TO RELATED APPLICATIONS

This application is a continuation of PCT International Application No. PCT/JP2015/079868 filed on Oct. 22, 2015 which claims the benefit of priority from Japanese Patent Application No. 2015-063108 filed on Mar. 25, 2015, the entire contents of which are incorporated herein by reference.

### BACKGROUND

1. Technical Field

The disclosure relates to a position detection system for detecting a position of a capsule medical device introduced into a subject. The disclosure also relates to a guidance system for guiding the capsule medical device.

2. Related Art

Conventionally, there has been developed capsule medical devices configured to be introduced into a subject to acquire various information related to inside of the subject or to administer medicine or the like in the subject. As an example, there has been known capsule endoscopes small enough to be introduced into a digestive canal (lumen) of the subject. The capsule endoscope has an imaging function and a wireless communication function inside a capsule-shaped casing. The capsule endoscope performs imaging while moving in the digestive canal after being swallowed by the subject and sequentially wirelessly transmits image data of images (hereinafter referred to as in-vivo images) of inside of an organ of the subject.

Systems for detecting a position of the capsule medical device in the subject have been developed. For example, JP 2008-132047 A discloses a position detection system in which a magnetic field generating coil that generates a magnetic field is provided in a capsule medical device, a sensing coil provided outside a subject detects the magnetic field generated by the magnetic field generating coil, and a position detection calculation of the capsule medical device is performed based on the strength of the detected magnetic field.

The detection accuracy of the capsule medical device introduced into the subject depends on the SN ratio of the magnetic field detected by the sensing coil and an arrangement condition of the sensing coil. Therefore, it is desired to realize an arrangement of the sensing coil that can reduce a position detection error of the capsule medical device as much as possible even when the SN ratio is low.

### SUMMARY

In some embodiments, a position detection system includes: a capsule medical device having therein a magnetic field generator configured to generate a magnetic field; a plurality of magnetic field detectors configured to detect the magnetic field generated by the magnetic field generator and output a plurality of detection signals; and a processor including hardware. The processor is configured to: calculate a position of the capsule medical device by using at least one of the plurality of detection signals respectively output by the plurality of magnetic field detectors; determine whether or not proper position detection of the capsule medical device based on the plurality of detection signals is possible by using a threshold, the threshold being a value based on at least one of output values of the plurality of detection signals respectively output by the plurality of magnetic field detectors under a predetermined condition; decide a determination value by using at least one of the output values of the plurality of detection signals, output from at least one of the plurality of magnetic field detectors; and determine that the proper position detection of the capsule medical device is not possible when the determination value is smaller than the threshold.

In some embodiments, a guidance system includes: the position detection system including the capsule medical device further having a permanent magnet; a guidance magnetic field generator configured to generate a magnetic field to be applied to the permanent magnet; and a guidance magnetic field controller configured to control the guidance magnetic field generator to perform guidance control for changing at least one of a position and a posture of the capsule medical device.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a configuration example of a guidance system according to a first embodiment of the present invention;

FIG. 2 is a schematic diagram illustrating an example of an internal structure of a capsule endoscope illustrated in FIG. 1;

FIG. 3 is a schematic diagram illustrating a configuration example of a guidance magnetic field generating device illustrated in FIG. 1;

FIG. 4 is a flowchart illustrating an operation of the guidance system illustrated in FIG. 1;

FIG. 5 is a schematic diagram for explaining a determination method performed by a determination unit;

FIG. 6 is a schematic diagram for explaining the determination method performed by the determination unit;

FIG. 7 is a schematic diagram for explaining a threshold setting method (1);

FIG. 8 is a schematic diagram illustrating an example of output values of detection signals detected by sensing coils;

FIG. 9 is a schematic diagram for explaining a threshold setting method (2);

FIG. 10 is a schematic diagram for explaining a determination value decision method in a second embodiment of the present invention;

FIG. 11 is a schematic diagram for explaining the determination value decision method in the second embodiment of the present invention;

FIG. 12 is a schematic diagram for explaining the determination value decision method in the second embodiment of the present invention; and

FIG. 13 is a schematic diagram illustrating an arrangement example of sensing coils in a third embodiment of the present invention.

### DETAILED DESCRIPTION

Hereinafter, a position detection system and a guidance system according to embodiments of the present invention will be described with reference to the drawings. The embodiments described below illustrate a capsule endoscope which is introduced into a subject through mouth and captures images in a subject (in a lumen) as one form of a capsule medical device to be detected by the position detection system. However, the present invention is not limited by the embodiments. In other words, it is possible to apply the present invention to position detection of various medical devices having a capsule shape, such as, for example, a capsule endoscope that moves in a lumen from the esophagus to the anus of the subject, a capsule medical device that delivers medicine or the like into the subject, and a capsule medical device including a PH sensor that measures PH in the subject.

In the description below, each drawing merely schematically illustrates shapes, sizes, and positional relationships in a degree such that contents of the present invention can be understood. Therefore, the present invention is not limited to the sizes, the shapes, and the positional relationships illustrated in each drawing. The same reference signs are used to designate the same elements throughout the drawings.

### First Embodiment

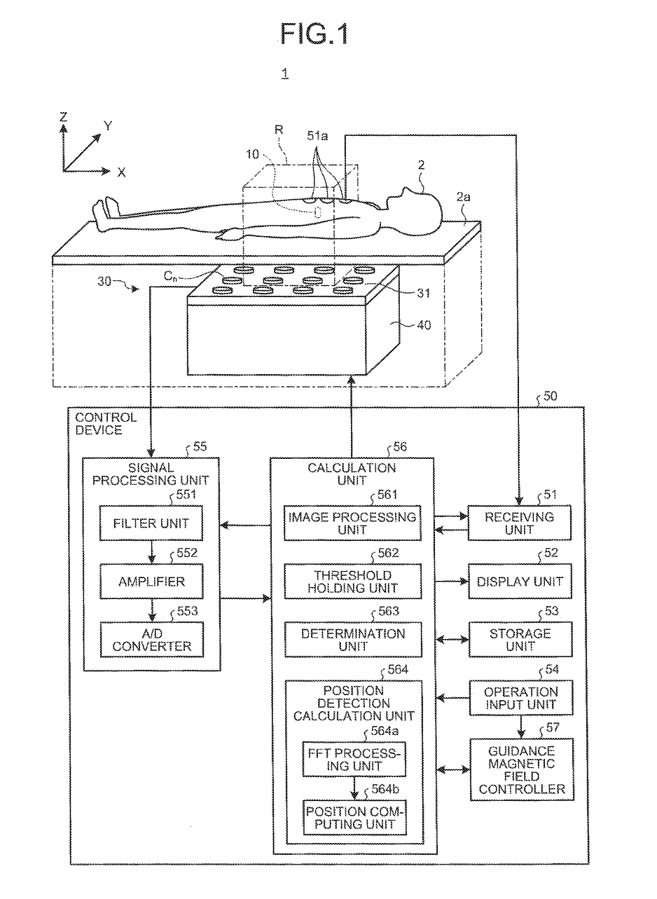

FIG. 1 is a schematic diagram illustrating a configuration example of a guidance system according to a first embodiment of the present invention. As illustrated in FIG. 1, the guidance system 1 according to the first embodiment includes a capsule endoscope 10 that transmits image data, which is acquired by capturing images of the inside of a subject 2, by superimposing the image data on a wireless signal, as an example of the capsule medical device to be introduced into a lumen of the subject 2, a magnetic field detection device 30 which is provided below a bed 2a, on which the subject 2 is mounted, and detects an alternating magnetic field generated by the capsule endoscope 10, a guidance magnetic field generating device 40 that generates a magnetic field for guiding the capsule endoscope 10, and a control device 50 that detects a position of the capsule endoscope 10 based on the alternating magnetic field detected by the magnetic field detection device 30 and guides the capsule endoscope 10 in the subject 2.

In the description below, un upper surface of the bed 2a, that is, a surface on which the subject 2 is mounted, is referred to as an XY plane (a horizontal plane), and a direction perpendicular to the XY plane is referred to as a Z direction (a vertical direction, that is, the gravity direction).

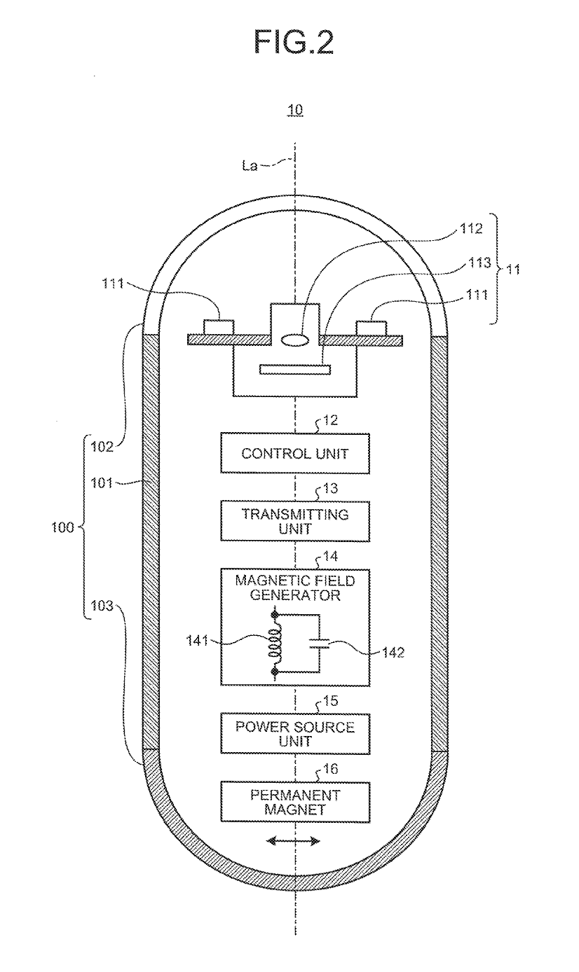

FIG. 2 is a schematic diagram illustrating an example of an internal structure of the capsule endoscope 10 illustrated in FIG. 1. As illustrated in FIG. 2, the capsule endoscope 10 includes a casing 100 having a capsule shape small enough to be easily introduced into a lumen of the subject 2, an imaging unit 11 that is housed in the casing 100 and captures an imaging signal by capturing images of the inside of the subject 2, a control unit 12 that controls operation of each element of the capsule endoscope 10 including the imaging unit 11 and performing predetermined signal processing on the imaging signal acquired by the imaging unit 11, a transmitting unit 13 that wirelessly transmits the imaging signal on which the signal processing is performed, a magnetic field generator 14 that generates an alternating magnetic field for detecting a position of the capsule endoscope 10, a power source unit 15 that supplies power to each element of the capsule endoscope 10, and a permanent magnet 16.

The casing 100 is an outer casing small enough to be introduced into the inside of an organ of the subject 2. The casing 100 includes a tubular casing 101 having a cylindrical shape and dome-shaped casings 102 and 103 having a dome shape. The casing 100 is realized by closing open ends of both sides of the tubular casing 101 with the dome-shaped casings 102 and 103 having a dome shape. The tubular casing 101 is formed of a colored material that is substantially opaque to visible light. At least one of the dome-shaped casings 102 and 103 (in FIG. 2, the dome-shaped casing 102 on the side of the imaging unit 11) is formed of an optical member that is transparent to light of a predetermined wavelength band such as visible light. In FIG. 2, the imaging unit 11 is provided only on the side of the dome-shaped casing 102. However, two imaging units 11 may be provided. In this case, the dome-shaped casing 103 is also formed of a transparent optical member. The casing 100 internally includes the imaging unit 11, the control unit 12, the transmitting unit 13, the magnetic field generator 14, the power source unit 15, and the permanent magnet 16 in a liquid-tight manner.

The imaging unit 11 has an illumination unit 111 such as an LED, an optical system 112 such as a condenser lens, and an image sensor 113 such as a CMOS image sensor or a CCD. The illumination unit 111 emits illumination light such as white light to an imaging visual field of the image sensor 113 and illuminates a subject in the imaging visual field through the dome-shaped casing 102. The optical system 112 collects reflection light from the imaging visual field on an imaging surface of the image sensor 113 and forms an image. The image sensor 113 converts the reflection light (optical signal) from the imaging visual field, which is received on the imaging surface, into an electrical signal and outputs the electrical signal as an image signal.

The control unit 12 causes the imaging unit 11 to operate at a predetermined imaging frame rate and causes the illumination unit 111 to emit light in synchronization with the imaging frame rate. The control unit 12 performs A/D conversion and other predetermined signal processing on an imaging signal generated by the imaging unit 11 and generates image data. Further, the control unit 12 causes the power source unit 15 to supply power to the magnetic field generator 14 and thereby causes the magnetic field generator 14 to generate an alternating magnetic field.

The transmitting unit 13 includes a transmitting antenna. The transmitting unit 13 acquires the image data on which the signal processing is performed by the control unit 12 and related information, performs modulation processing on the image data and the related information, and sequentially wirelessly transmits the image data and the related information to the outside through the transmitting antenna.

The magnetic field generator 14 includes a magnetic field generating coil 141 that forms a part of a resonance circuit and generates a magnetic field when a current flows and a capacitor 142 that forms the resonance circuit along with the magnetic field generating coil 141. The magnetic field generator 14 receives power supply from the power source unit 15 and generates an alternating magnetic field of a predetermined frequency.

The power source unit 15 is a power storage unit such as a button-type battery or a capacitor and has a switch unit such as a magnetic switch or an optical switch. If the power source unit 15 has a magnetic switch, the power source unit 15 switches between ON and OFF of a power source by a magnetic field applied from outside. When the power source is in an ON state, the power source unit 15 appropriately supplies power of the power storage unit to each element (the imaging unit 11, the control unit 12, and the transmitting unit 13) of the capsule endoscope 10. When the power source is in an OFF state, the power source unit 15 stops power supply to each element of the capsule endoscope 10.

The permanent magnet 16 allows magnetic guidance of the capsule endoscope 10 by the magnetic field generated by the guidance magnetic field generating device 40. The permanent magnet 16 is fixedly arranged inside the casing 100 having a capsule shape so that a magnetization direction has an inclination with respect to a long axis La of the casing 100. In FIG. 2, the magnetization direction of the permanent magnet 16 is indicated by an arrow. In the first embodiment, the permanent magnet 16 is arranged so that the magnetization direction is perpendicular to the long axis La. The permanent magnet 16 moves following a magnetic field applied from outside. As a result, the magnetic guidance of the capsule endoscope 10 by the guidance magnetic field generating device 40 is realized.

Referring to FIG. 1 again, the magnetic field detection device 30 includes a planner panel 31 and a plurality of sensing coils Cₙ (n=1, 2, . . . , and so on) which are arranged on a main surface of the panel 31 and each of which receives the alternating magnetic field generated from the capsule endoscope 10 and outputs a detection signal. Each sensing coil Cₙ is a magnetic field detector composed of a cylindrical coil in which a coil wire is wound in a coil spring shape, and, for example, has an opening diameter of about 30 to 40 mm and a height of about 5 mm.

The magnetic field detection device 30 as described above is arranged near the subject 2 that is being examined. In the first embodiment, the magnetic field detection device 30 is arranged below the bed 2a and the main surface of the panel 31 is arranged to be horizontal.

An area where the position of the capsule endoscope 10 can be detected by the magnetic field detection device 30 is a detection target area R. The detection target area R is a three-dimensional closed area including a range in which the capsule endoscope 10 can move in the subject 2 (that is, a range of an organ to be observed). The detection target area R is set in advance according to an arrangement of the plurality of coils Cₙ in the magnetic field detection device 30 and the strength of a magnetic field that can be generated by the magnetic field generator 14 in the capsule endoscope 10.

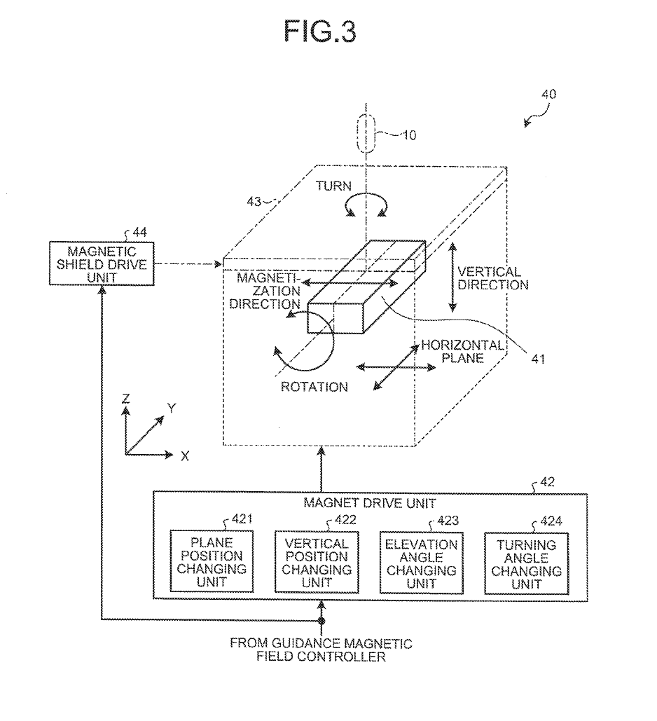

FIG. 3 is a schematic diagram illustrating a configuration example of the guidance magnetic field generating device 40. As illustrated in FIG. 3, the guidance magnetic field generating device 40 generates a magnetic field for relatively changing the position of the capsule endoscope 10 introduced into the subject 2, an inclination angle and an azimuth angle of the long axis La with respect to the vertical direction, with respect to the subject 2. More specifically, the guidance magnetic field generating device 40 includes an extracorporeal permanent magnet 41 as a guidance magnetic field generator (a second magnetic field generator) that generates a magnetic field, a magnet drive unit 42 that changes the position and the posture of the extracorporeal permanent magnet 41, and a magnetic shield 43 and a magnetic shield drive unit 44 constituting a shield device configured to shield the magnetic field generated by the extracorporeal permanent magnet 41. Among them, the magnet drive unit 42 includes a plane position changing unit 421, a vertical position changing unit 422, an elevation angle changing unit 423, and a turning angle changing unit 424.

The extracorporeal permanent magnet 41 is preferably realized by a bar magnet having a rectangular parallelepiped shape and constrains the capsule endoscope 10 inside an area where one of four surfaces of the extracorporeal permanent magnet 41 in parallel with a magnetization direction of the extracorporeal permanent magnet 41 is projected on a horizontal plane. Instead of the extracorporeal permanent magnet 41, an electromagnet that generates a magnetic field when a current flows through it may be provided.

The magnet drive unit 42 operates according to a control signal output from a guidance magnetic field controller 57 described later. Specifically, the plane position changing unit 421 translates the extracorporeal permanent magnet 41 in an XY plane. In other words, the extracorporeal permanent magnet 41 is moved into a horizontal plane in a state in which relative positions of two magnetic poles magnetized in the extracorporeal permanent magnet 41 are secured.

The vertical position changing unit 422 translates the extracorporeal permanent magnet 41 along the Z direction. In other words, the extracorporeal permanent magnet 41 is moved along the vertical direction in a state in which relative positions of two magnetic poles magnetized in the extracorporeal permanent magnet 41 are secured.

The elevation angle changing unit 423 changes an angle of the magnetization direction of the extracorporeal permanent magnet 41 with respect to the horizontal plane by rotating the extracorporeal permanent magnet 41 in a vertical plane including the magnetization direction.

The turning angle changing unit 424 turns the extracorporeal permanent magnet 41 with respect to a vertical direction axis passing through the center of the extracorporeal permanent magnet 41.

The magnetic shield 43 is a member formed by a ferromagnetic body such as iron or nickel and is insertably and removably provided at least above the extracorporeal permanent magnet 41. The magnetic shield drive unit 44 performs insertion and removal of the magnetic shield 43 according to a control signal output from the guidance magnetic field controller 57 described later. While the magnetic shield 43 is removed from above the extracorporeal permanent magnet 41, a magnetic field is generated in a space including the detection target area R by the extracorporeal permanent magnet 41. During this period, a guidance of the capsule endoscope 10 can be performed by the guidance magnetic field generating device 40. On the other hand, while the magnetic shield 43 is inserted above the extracorporeal permanent magnet 41, the magnetic field generated by the extracorporeal permanent magnet 41 is shielded inside the guidance magnetic field generating device 40. That is, the guidance of the capsule endoscope 10 is not performed during this period.

If an electromagnet is provided instead of the extracorporeal permanent magnet 41, it is not necessary to provide the magnetic shield 43 and the magnetic shield drive unit 44. In this case, when power supply to the electromagnet is stopped, the generation of magnetic field from the guidance magnetic field generating device 40 is stopped, so that a power controller for controlling the power supply to the electromagnet functions as a shield device for the magnetic field.

Referring to FIG. 1 again, the control device 50 includes a receiving unit 51 that receives a wireless signal transmitted from the capsule endoscope 10 through a receiving antenna 51a, a display unit 52 that outputs various information processed by the control device 50 to a display device and causes the display device to display the various information. a storage unit 53, an operation input unit 54 used for inputting various information and commands into the control device 50, a signal processing unit 55 that performs various signal processing on a detection signal output from each sensing coil Cₙ and generates magnetic field information, a calculation unit 56 that performs various calculation processing such as image generation based on image data received by the receiving unit 51 and position detection of the capsule endoscope 10 based on the magnetic field information generated by the signal processing unit 55, and the guidance magnetic field controller 57 that performs control for guiding the capsule endoscope 10.

When performing examination by using the capsule endoscope 10, a plurality of receiving antennas 51a that receive a wireless signal transmitted from the capsule endoscope 10 are attached to a body surface of the subject 2. The receiving unit 51 selects a receiving antenna 51a whose receiving strength of the wireless signal is the highest from among the receiving antennas 51a and performs demodulation processing and the like on the wireless signal received through the selected receiving antenna 51a, and thereby acquires image data of an in-vivo image and related information.

The display unit 52 includes various displays such as a liquid crystal and an organic EL and displays various information input from the operation input unit 54, the in-vivo image of the subject 2, position information of the capsule endoscope 10 of when the in-vivo image is captured, and the like.

The storage unit 53 is realized by using a storage medium such as a flash memory and a hard disk which rewritably stores information and a write/read device. The storage unit 53 stores various programs and various parameters for the calculation unit 56 to control each unit of the control device 50, image data of an in-vivo image captured by the capsule endoscope 10, position information of the capsule endoscope 10 in the subject 2, and the like.

The operation input unit 54 is realized by input devices such as various buttons, switches, and a keyboard, pointing devices such as a mouse and a touch panel, a joystick, and the like. The operation input unit 54 inputs various information into the calculation unit 56 according to an input operation performed by a user. The information input by the operation input unit 54 includes, for example, information for guiding the capsule endoscope 10 to position and posture desired by the user (hereinafter referred to as guidance operation information).

The signal processing unit 55 includes a filter unit 551 that shapes a waveform of a detection signal output from the magnetic field detection device 30, an amplifier 552, and an A/D converter 553 that performs A/D conversion processing on the detection signal. Although there are an alternating magnetic field generated by the magnetic field generator 14 in the capsule endoscope 10 and a guidance magnetic field formed by the guidance magnetic field generating device 40 in a space where the magnetic field detection device 30 can detect a magnetic field, both magnetic fields have frequencies completely different from each other, so that there is no interference between the magnetic fields.

The calculation unit 56 is configured by using, for example, a CPU (Central Processing Unit) and the like. The calculation unit 56 reads a program from the storage unit 53 and integrally controls operation of the control device 50 by transmitting instructions and data to each unit included in the control device 50. The calculation unit 56 includes an image processing unit 561, a threshold holding unit 562, a determination unit 563, and a position detection calculation unit 564.

The image processing unit 561 generates display image data by performing predetermined image processing such as white balance processing, demosaicing, gamma conversion, smoothing (noise removal and the like), and the like on image data input from the receiving unit 51.

The determination unit 563 determines whether or not to cause the position detection calculation unit 564 described below to perform a position detection calculation of the capsule endoscope 10 based on an output value of a detection signal output from the signal processing unit 55. The threshold holding unit 562 holds a threshold used for the determination.

When the determination unit 563 determines to cause the position detection calculation unit 564 to perform the position detection calculation, the position detection calculation unit 564 acquires information (position information) representing the position of the capsule endoscope 10 based on the detection signal output from the signal processing unit 55. More specifically, the position detection calculation unit 564 includes an FFT processing unit 564a that extracts magnetic field information such as amplitude and phase of the alternating magnetic field by performing fast Fourier transform processing (hereinafter referred to as FFT processing) on detection data output from the signal processing unit 55 and a position computing unit 564b that calculates the position of the capsule endoscope 10 based on the magnetic field information extracted by the FFT processing unit 564a.

The capsule endoscope 10, the magnetic field detection device 30, the signal processing unit 55, the threshold holding unit 562, the determination unit 563, and the position detection calculation unit 564 of the guidance system 1 illustrated in FIG. 1 constitute the position detection system.

The guidance magnetic field controller 57 controls operation of each unit of the magnet drive unit 42 so that the capsule endoscope 10 has a posture desired by the user at a position desired by the user based on the position and posture of the capsule endoscope 10 calculated by the position detection calculation unit 564 and the guidance operation information input from the operation input unit 54. Specifically, the guidance magnetic field controller 57 guides the capsule endoscope 10 by changing a magnetic gradient in a space including the position of the capsule endoscope 10 by changing the position, elevation angle, and turning angle of the extracorporeal permanent magnet 41.

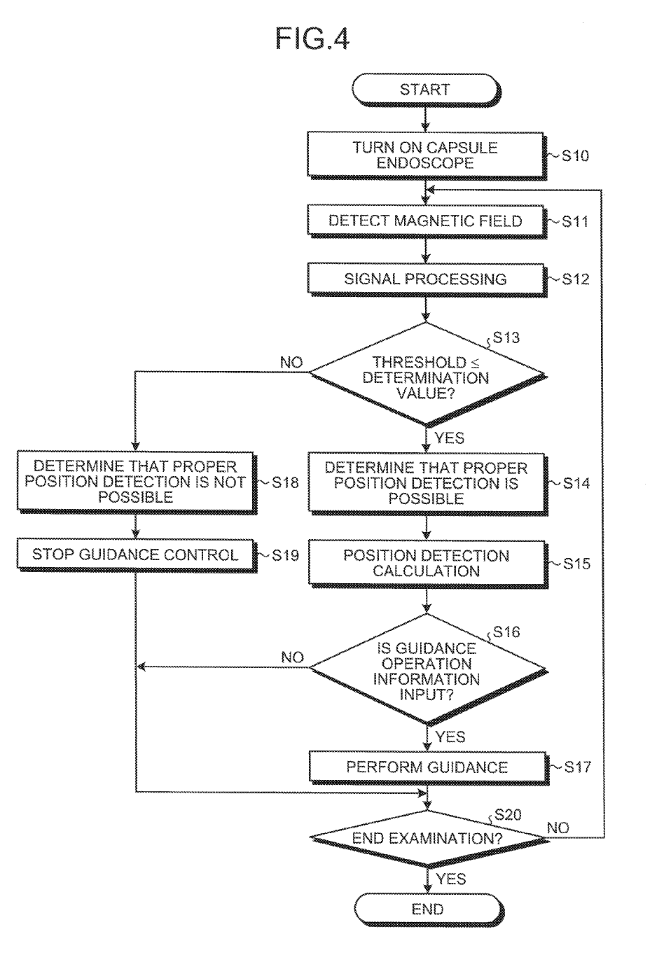

Next, an operation of the guidance system 1 will be described. FIG. 4 is a flowchart illustrating the operation of the guidance system 1.

First, in step S10, the capsule endoscope 10 is turned on. Thereby, power supply to each unit of the capsule endoscope 10 is started from the power source unit 15 (see FIG. 2), so that the imaging unit 11 starts imaging and the magnetic field generator 14 starts generation of magnetic field.

In step S11, the magnetic field detection device 30 detects the magnetic field. Specifically, each sensing coil Cₙ of the magnetic field detection device 30 generates a current according to a magnetic field distributed at a position of the sensing coil Cₙ and outputs the current to the signal processing unit 55 as a detection signal of the magnetic field.

In step S12, the signal processing unit 55 receives a plurality of detection signals output from the magnetic field detection device 30 (currents respectively generated by a plurality of sensing coils Cₙ), performs signal processing such as waveform shaping, amplification, and A/D conversion on the detection signals, and then outputs the detection signals.

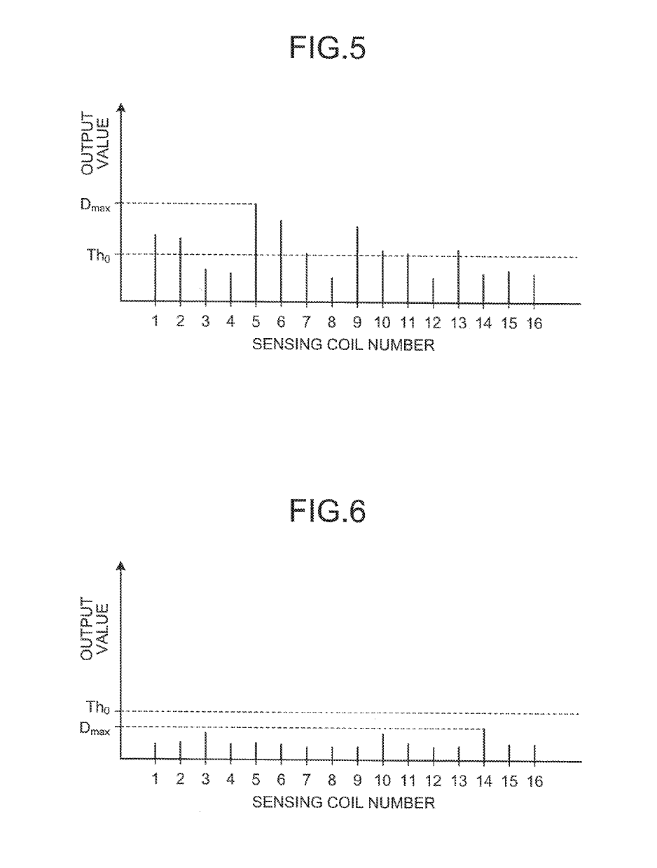

In step S13, the determination unit 563 compares a threshold held in advance by the threshold holding unit 562 with a determination value decided based on output values (amplitudes) of a plurality of detection signals output from the signal processing unit 55. A setting method of the threshold and a decision method of the determination value will be described later in detail. FIGS. 5 and 6 are schematic diagrams for explaining a determination method of output values of the sensing coils Cₙ. Here, as an example, as illustrated in FIGS. 5 and 6, the maximum value Dₘₐₓ of the output values of a plurality of sensing coils Cₙ is used as a determination value and it is determined whether or not the determination value Dₘₐₓ is greater than or equal to a threshold Th₀.

As illustrated in FIG. 5, when the determination value (the maximum value Dₘₐₓ) is greater than or equal to the threshold Th₀ (step S13: Yes), the determination unit 563 determines that the capsule endoscope 10 really exists in the detection target area R and proper position detection is possible (step S14). Here, “proper position detection is possible” means that signals detected by the sensing coils Cₙ include a magnetic field component generated by the capsule endoscope 10 and a position detection calculation based on the magnetic field component is possible. On the other hand, “proper position detection is not possible” means that signals detected by the sensing coils Cₙ do not include sufficient magnetic field component generated by the capsule endoscope 10 and a position detection calculation based on noise components is performed.

In this case, the position detection calculation unit 564 performs a position detection calculation of the capsule endoscope 10 based on the plurality of detection signals output from the signal processing unit 55 (step S15). Specifically, the FFT processing unit 564a calculates amplitude and phase of the detection signals by performing fast Fourier transform processing on each detection signal. The amplitude and phase correspond to the strength and phase of a magnetic field at a position of each sensing coil Cₙ. The position computing unit 564b calculates the position and posture of the capsule endoscope 10 based on the amplitude and phase of the detection signals.

In the following step S16, the guidance magnetic field controller 57 determines whether or not guidance operation information is input from the operation input unit 54. When the guidance operation information is input (step S16: Yes), the guidance magnetic field controller 57 performs guidance of the capsule endoscope 10 by controlling an operation of the guidance magnetic field generating device 40 based on the guidance operation information and the position and posture of the capsule endoscope 10 calculated in step S15 (step S17).

On the other hand, when the guidance operation information is not input from the operation input unit 54 (step S16: No), the operation of the guidance system 1 directly proceeds to step S20.

In step S13, as illustrated in FIG. 6, when the determination value Dₘₐₓ is smaller than the threshold Th₀ (step S13: No), the determination unit 563 determines that the capsule endoscope 10 does not exist in the detection target area R and proper position detection is not possible (step S18). In this case, the position detection calculation unit 564 does not perform the position detection calculation of the capsule endoscope 10 and the operation proceeds to the following step S19.

In step S19, the guidance magnetic field controller 57 stops guidance control on the capsule endoscope 10. Specifically, the guidance magnetic field controller 57 performs control on the magnetic shield drive unit 44 of the guidance magnetic field generating device 40 to insert the magnetic shield 43 above the extracorporeal permanent magnet 41 and shield the magnetic field generated by the extracorporeal permanent magnet 41 inside the guidance magnetic field generating device 40. Thereby, even when the guidance operation information is input from the operation input unit 54, the guidance magnetic field is not applied to the capsule endoscope 10. Thereafter, the operation of the guidance system 1 proceeds to step S20.

In step S20, the control device 50 determines whether or not to end the examination performed by using the capsule endoscope 10. Specifically, the control device 50 determines to end the examination when an instruction signal to end the examination is input through the operation input unit 54 or a predetermined period of time or more has elapsed since the power of the capsule endoscope 10 was turned on.

In ending the examination (step S20: Yes), the operation of the guidance system 1 ends. On the other hand, if not ending the examination (step S20: No), the operation of the guidance system 1 returns to step S11.

Next, the setting method of the threshold used in step S13 will be described. The setting method of the threshold includes setting methods (1) and (2) described below. In step S13 described above, it is possible to use a threshold acquired by any of the threshold setting methods (1) and (2).

Threshold Setting Method (1)

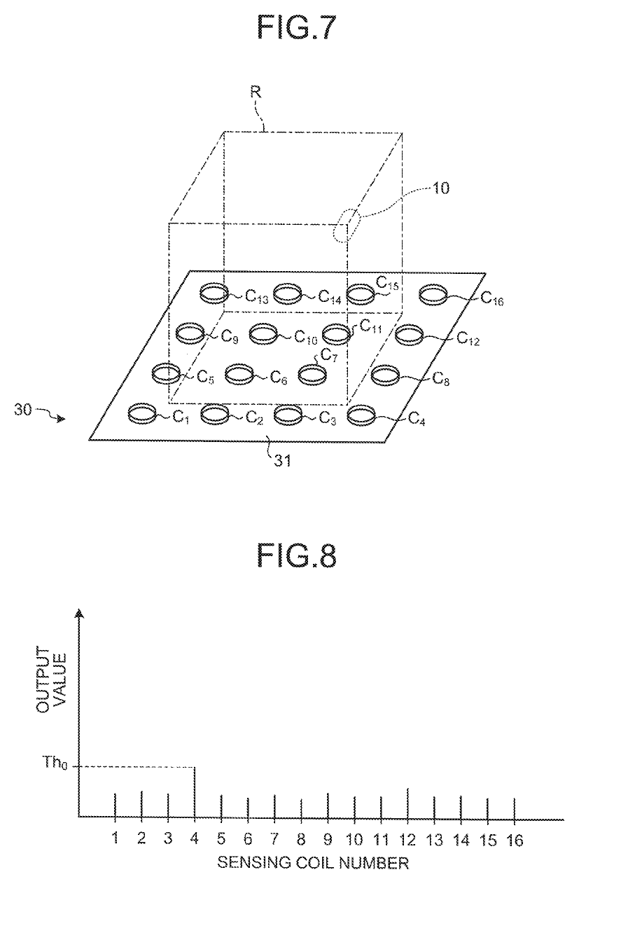

FIG. 7 is a schematic diagram for explaining the threshold setting method (1) and illustrates a plurality of sensing coils Cₙ (as an example, n=1 to 16) provided in the magnetic field detection device 30 and the detection target area R of the capsule endoscope 10.

The threshold is acquired before starting the examination performed by using the capsule endoscope 10. First, the power of the capsule endoscope 10 is turned on to cause the magnetic field generator 14 to generate a magnetic field. Then, the capsule endoscope 10 generating the magnetic field is located where detection level of each sensing coil Cₙ with respect to the magnetic field generated by the capsule endoscope 10 is the lowest, in other words, located at an end portion of the detection target area R (a boundary surface or a boundary line), preferably at a corner which is an endmost portion. FIG. 7 illustrates a state in which the capsule endoscope 10 is arranged to one of the four corners of the upper surface of the detection target area R.

FIG. 8 is a schematic diagram illustrating an example of output values of detection signals detected by the sensing coils Cₙ in this state. In FIG. 8, the output value of the sensing coil C₄ is the greatest. In this case, the greatest output value is acquired as the threshold Th₀.

Threshold Setting Method (2)

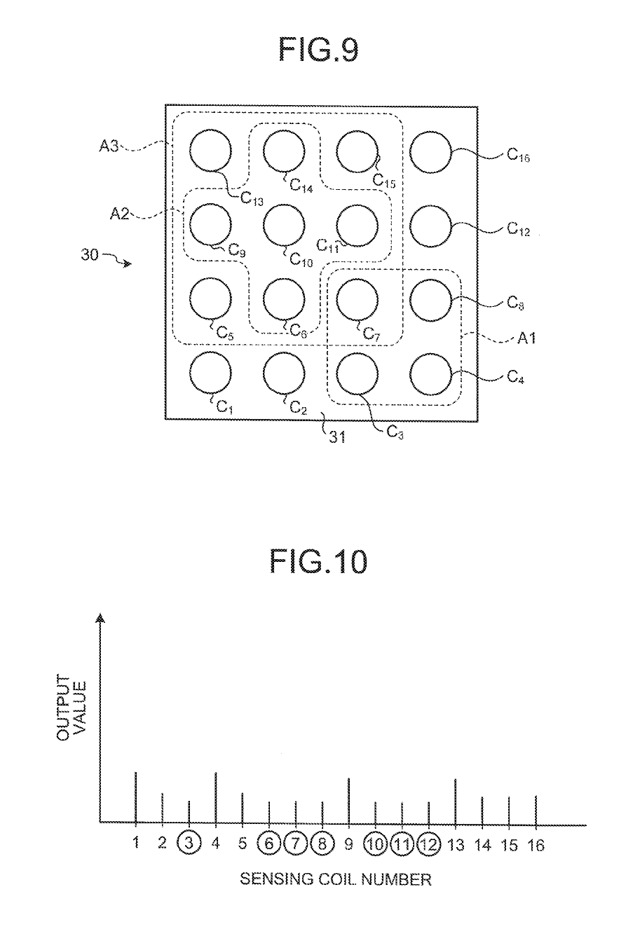

In the same manner as in the threshold setting method (1), the capsule endoscope 10 generating the magnetic field is located where detection level of each sensing coil Cₙ with respect to the magnetic field generated by the capsule endoscope 10 is the lowest (an end portion of the detection target area R) (see FIG. 7). Then, an average value of output values of a sensing coil Cₙ whose output value is the greatest and a predetermined number of (one or more) sensing coils Cₙ located near the sensing coil Cₙ whose output value is the greatest is set as the threshold. FIG. 9 is a schematic diagram for explaining the threshold setting method (2).

For example, in the case of FIG. 8, the output value of the sensing coil C₄ is the greatest, and as illustrated in FIG. 9, sensing coils Cₙ located near the sensing coil C₄ are sensing coils C₃, C₇, and C₈. Therefore, an average value of output values of a sensing coil group (sensing coils C₃, C₄, C₇, and C₈) included in a range A1 is set as the threshold.

Here, the sensing coil Cₙ whose output value is the greatest and the sensing coils Cₙ located near the sensing coil Cₙ whose output value is the greatest are not limited to four sensing coils in a corner of a sensing coil group arranged on the panel 31 as in the range A1. As another example, when the output value of the sensing coil C₁₀ is the greatest, four sensing coils C₆, C₉, C₁₁, and C₁₄ adjacent to the sensing coil C₁₀ in the vertical direction and the horizontal direction may be defined as the sensing coils Cₙ located near the sensing coil C₁₀. In this case, an average value of output values of a sensing coil group included in a range A2 is set as the threshold. As further another example, eight sensing coils C₅, C₆, C₇, C₉, C₁₁, C₁₃, C₁₄, and C₁₅ adjacent to the sensing coil C₁₀, whose output value is the greatest, in the vertical direction, the horizontal direction, and the diagonal directions may be defined as the sensing coils Cₙ located near the sensing coil C₁₀. In this case, an average value of output values of a sensing coil group included in a range A3 is set as the threshold.

These thresholds may be calculated in advance based on a theoretical value. Specifically, it is assumed that a magnetic field generating source that generates a magnetic field of predetermined strength (similar to that of the magnetic field generated by the capsule endoscope 10) is arranged at an end portion of the detection target area R, and the threshold is calculated in the same manner as in the threshold setting method (1) or (2) described above based on a theoretical value of the strength of the magnetic field at a position of each sensing coil Cₙ.

Next, a decision method of the determination value to be compared with the threshold in step S13 will be described. A determination value decision method includes decision methods (1) to (4) described below. In step S13 described above, it is possible to use a determination value decided by any of the determination value decision methods (1) to (4).

Determination Value Decision Method (1)

As described in step S13 described above, the maximum value of the output values of a plurality of sensing coils Cₙ is defined as the determination value. For example, in the case of FIG. 5, the output value of the sensing coil C₅ is the greatest, so that the maximum value Dₘₐₓ is decided as the determination value and is compared with the threshold Th₀.

Determination Value Decision Method (2)

an average value of a predetermined number of (two or more) output values selected in descending order of value from the output values of a plurality of sensing coils Cₙ is defined as the determination value. For example, when an average value of four output values selected in descending order of value is defined as the determination value, if the output values illustrated in FIG. 5 are obtained, an average value of output values of the sensing coils C₁, C₅, C₆, and C₉ is decided as the determination value.

Here, an improper position (ghost) of the capsule endoscope 10 that can be detected in a conventional position detection system depends on noise distribution, so that the position of the detected ghost and a signal level are substantially constant. Therefore, output values of a plurality of sensing coils Cₙ whose output values tend to be large are used for the determination value. Therefore, it is possible to accurately determine whether current output values from the sensing coils Cₙ are a detection result of the magnetic field generated by the capsule endoscope 10 or a detection result of high level noise.

Determination Value Decision Method (3)

An output value of a sensing coil Cₙ whose output value is the greatest among a plurality of sensing coils Cₙ and output values of one or more sensing coils Cₙ adjacent to the sensing coil Cₙ whose output value is the greatest are defined as the determination values. For example, in the case of FIG. 5, the output value of the sensing coil C₅ is the greatest, so that the output value of the sensing coil C₅ and the output value of any one of the sensing coils C₁, C₆, and C₉ (see FIG. 9) adjacent to the sensing coil C₅ are defined as the determination values. In this case, the output value of the sensing coil C₅ and the output value of the adjacent sensing coil Cₙ are greater than or equal to the threshold Th₀, it is determined that proper position detection is possible. The sensing coil Cₙ from which the determination value is acquired may be decided in advance from among the adjacent coils C₁, C₆, and C₉, or an output value of a sensing coil Cₙ located in a moving direction of the capsule endoscope 10 may be used as the determination value.

Determination Value Decision Method (4)

An average value of output values of a sensing coil Cₙ whose output value is the greatest among a plurality of sensing coils Cₙ and sensing coils Cₙ located near the sensing coil Cₙ whose output value is the greatest is defined as the determination value. For example, in the case of FIG. 5, the output value of the sensing coil C₅ is the greatest, so that an average value of output values of the sensing coil C₅ and the sensing coils C₁, C₂, C₆, C₉, and C₁₀ located near the sensing coil C₅ (see FIG. 9) is decided as the determination value and is compared with the threshold Th₀. As the sensing coils Cₙ located near the sensing coil Cₙ whose output value is the greatest, it is possible to select a sensing coil group located adjacent to the sensing coil Cₙ whose output value is the greatest in the vertical direction and the horizontal direction or a sensing coil group located adjacent to the sensing coil Cₙ whose output value is the greatest in the vertical direction, the horizontal direction, and the diagonal directions.

Here, when the capsule endoscope 10 actually exists in the detection target area R, if there is a sensing coil Cₙ whose output value is large, output values of sensing coils Cₙ around the sensing coil Cₙ whose output value is large tend to be large. On the other hand, when the capsule endoscope 10 does not exist in the detection target area R, even if there is a sensing coil Cₙ whose output value is large, output values of sensing coils Cₙ located near the sensing coil Cₙ whose output value is large are not necessarily large. Therefore, it is possible to accurately determine whether or not the capsule endoscope 10 exists in the detection target area R by comparing an average value of output values of the sensing coil Cₙ whose output value is the greatest and sensing coils Cₙ located near the sensing coil Cₙ whose output value is the greatest with the threshold Th₀.

As described above, in the first embodiment of the present invention, the determination value decided based on the output values of the sensing coils Cₙ is compared with the threshold acquired in advance, and it is determined whether or not proper position detection of the capsule endoscope 10 is possible based on a result of the comparison. When it is determined that proper position detection is not possible, the position detection calculation is not performed by the position detection calculation unit 564, so that it is possible to prevent improper output of a position detection result of the capsule endoscope 10.

Further, according to the first embodiment of the present invention, when it is not possible to perform proper position detection of the capsule endoscope 10, the guidance control on the capsule endoscope 10 is turned off, so that it is possible to prevent improper guidance based on an erroneously detected position of the capsule endoscope 10.

### Modified Example 1

Next, a modified example 1 of the first embodiment of the present invention will be described.

In the threshold setting method (2) described in the first embodiment, the capsule endoscope 10 is located where the detection level of each sensing coil Cₙ with respect to the magnetic field generated by the capsule endoscope 10 is the lowest and the average value of output values of the sensing coil Cₙ whose output value is the greatest and a predetermined number of sensing coils Cₙ located near the sensing coil Cₙ whose output value is the greatest is set as the threshold. However, the sum of the output values may be set as the threshold.

In this case, although the average value of a plurality of output values is defined as the determination value in the determination value decision methods (2) and (4) described above, the sum of the output values is decided as the determination value. On this occasion, the sensing coils Cₙ from which the output values used to decide the determination value are selected so that the number of output values used to acquire the threshold corresponds to the number of output values used to decide the determination value.

### Second Embodiment

Next, a second embodiment of the present invention will be described. The configuration and the operation of the guidance system according to the second embodiment is almost the same as those of the first embodiment (see FIGS. 1 and 4). However, the decision method of the determination value to be compared with the threshold is different from that of the first embodiment.

In the first embodiment described above, output values are acquired from all the sensing coils Cₙ and the determination value is decided based on the output values. However, the sensing coils Cₙ, from which the output values used to decide the determination value to be compared with the threshold are acquired, may be selected in advance at a time of calibration performed before the examination performed by using the capsule endoscope 10. In other words, in a state in which the capsule endoscope 10 does not generate magnetic field and there is no effect of magnetic field generated by the magnetic field generator 14 in the detection target area R, detection signals from the sensing coils Cₙ are acquired, and sensing coils Cₙ in which noise level is low are selected in advance as sensing coils Cₙ from which the determination value is acquired. Regarding a selection method of the sensing coils Cₙ, a predetermined number of (one or more) sensing coils Cₙ may be selected in ascending order from the sensing coil Cₙ in which the noise level is the lowest, or all sensing coils Cₙ in which the noise level is lower than or equal to a predetermined value may be selected. Therefore, all the sensing coils Cₙ arranged on the panel 31 can be selected, or only one sensing coil Cₙ may be selected. In the latter case, the output value of the selected sensing coil Cₙ is directly used as the determination value.

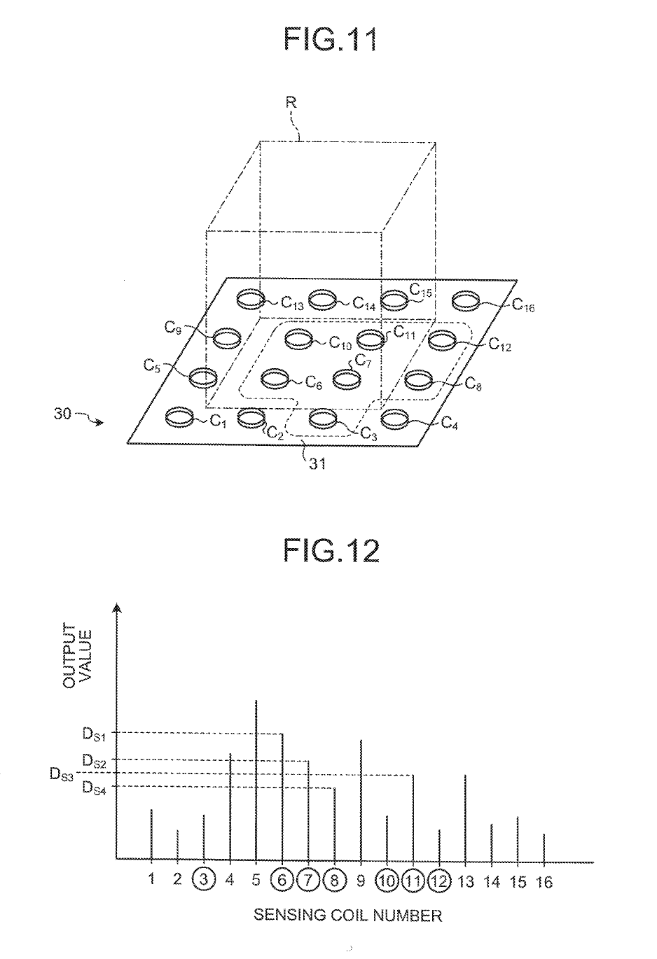

FIGS. 10 to 12 are schematic diagrams for explaining the determination value decision method in the second embodiment. For example, at a time of calibration before the examination, the output values (noise levels) of the sensing coils Cₙ as illustrated in FIG. 10 are obtained, and sensing coils C₃, C₆, C₇, C₈, C₁₀, C₁₁, and C₁₂ are selected as sensing coils Cₙ from which the determination value is acquired (see FIG. 11). In step S13, the determination value is decided based on the output values of the selected sensing coils C₃, C₆, C₇, C₈, C₁₀, C₁₁, and C₁₂. The circled numbers illustrated in FIGS. 10 and 12 are coil numbers of the selected sensing coils Cₙ.

As an example of the determination value decision method, the maximum value among the output values of sensing coils C₃, C₆, C₇, C₈, C₁₀, C₁₁, and C₁₂ selected in advance is defined as the determination value. For example, after starting the examination performed by using the capsule endoscope 10, when the output values of the sensing coils Cₙ illustrated in FIG. 12 are obtained, the output values Dₛ₁ of the sensing coil C₆ is the greatest among the output values of the sensing coils C₃, C₆, C₇, C₈, C₁₀, C₁₁, and C₁₂. Therefore, the output value Dₛ₁ is decided as the determination value and is compared with the threshold.

### Modified Example 2-1

Next, a modified example 2-1 of the second embodiment of the present invention will be described.

As another example of the determination value decision method, a predetermined number of (two or more) output values selected in descending order of value from the output values of sensing coils C₃, C₆, C₇, C₈, C₁₀, C₁₁, and C₁₂ selected in advance may be defined as the determination values. For example, when the largest and the second largest output values are defined as the determination values, in FIG. 12, the output value Dₛ₁ of the sensing coil C₆ and the output value Dₛ₂ of the sensing coil C₇ are decided as the determination values. In this case, each of the output value Dₛ₁ and the output value Dₛ₂ is compared with the threshold, and when both output values are greater than or equal to the threshold, it is determined that proper position detection of the capsule endoscope 10 is possible.

### Modified Example 2-2

Next, a modified example 2-2 of the second embodiment of the present invention will be described.

As further another example of the determination value decision method, an average value of a predetermined number of (two or more) output values selected in descending order of value from the output values of sensing coils C₃, C₆, C₇, C₈, C₁₀, C₁₁, and C₁₂ selected in advance may be defined as the determination values. For example, when an average value of four output values selected in descending order of value is defined as the determination value, in FIG. 12, an average value of the output value Dₛ₁ of the sensing coil C₆, the output value Dₛ₂ of the sensing coil C₇, the output value Dₛ₃ of the sensing coil C₁₁, and the output value Dₛ₄ of the sensing coil C₈ is decided as the determination value. Alternatively, the sum of the output values Dₛ₁D₃₂, Dₛ₃, and Dₛ₄ may be decided as the determination value.

### Modified Example 2-3

Next, a modified example 2-3 of the second embodiment of the present invention will be described.

As further another example of the determination value decision method, an average value of output values of a sensing coil Cₙ whose output value is the greatest among the sensing coils C₃, C₆, C₇, C₈, C₁₀, C₁₁, and C₁₂ selected in advance and sensing coils Cₙ located near the sensing coil Cₙ whose output value is the greatest may be defined as the determination value. For example, in FIG. 12, the output value of the sensing coil C₆ is the greatest among the sensing coils C₃, C₆, C₇, C₈, C₁₀, C₁₁, and C₁₂, so that an average value of output values of the sensing coil C₆ and the sensing coils C₃, C₇, and C₁₀ located near the sensing coil C₆ (see FIG. 11) is decided as the determination value. Alternatively, the sum of the output values may be decided as the determination value.

As described in the second embodiment and the modified examples 2-1 to 2-3 described above, when the determination value is decided at a time of calibration based on the output values of the sensing coils Cₙ selected in advance, the threshold is acquired based on a theoretical value of the output value of each sensing coil Cₙ under a condition that the detection level of each sensing coil Cₙ with respect to the magnetic field generated by the capsule endoscope 10 is the lowest (a condition that the capsule endoscope 10 is arranged at an end portion of the detection target area R). A setting method of the threshold based on a theoretical value is the same as the threshold setting methods (1) and (2) described in the first embodiment or the threshold setting method described in the modified example 1.

### Third Embodiment

Next, a third embodiment of the present invention will be described.

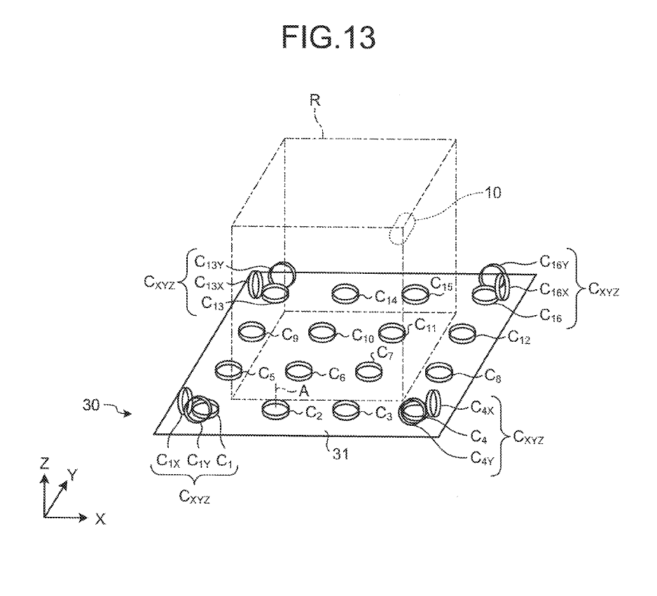

In the first and the second embodiments described above, the orientation of all the sensing coils Cₙ arranged on the panel 31 is aligned. However, the orientations of some sensing coils Cₙ may be changed. FIG. 13 is a schematic diagram illustrating an arrangement example of the sensing coils Cₙ in the third embodiment of the present invention. FIG. 13 illustrates an example in which rotation center axes A of the sensing coils Cₙ are aligned to one of three directions perpendicular to each other (XYZ directions).

In FIG. 13, the sensing coils C₁ to C₁₆ are arranged so that the rotation center axes A are in parallel with Z axis (that is, apertures of the coils are in parallel with an XY plane) and sensing coils C_{1X}, C_{4X}, C_{13X}, and C_{16X} whose rotation center axes A are in parallel with X axis and sensing coils C_{1Y}, C_{4Y}, C_{13Y}, and C_{16Y} whose rotation center axes A are in parallel with Y axis are arranged close to sensing coils C₁, C₄, C₁₃, and C₁₆, respectively, arranged at end portions of the panel 31. The shape and the size of the sensing coils C_{1X}, C_{4X}, C_{13X}, C_{16X}, C_{1Y}, C_{4Y}, C_{13Y}, and C_{16Y} are the same as those of the sensing coils C₁ to C₁₆.

Here, the sensing coils Cₙ (n=1 to 16, 1X, 1Y, 4X, 4Y, 13X, 13Y, 16X, and 16Y) can accurately detect a change in the magnetic field in a direction in parallel with the rotation center axis A. Therefore, three sensing coils (for example, the sensing coils C₁, C_{1X}, and C_{1Y}) that are arranged so that their rotation center axes A are in parallel with Z axis, X axis, and Y axis, respectively, are arranged close to each other as one unit (as a coil set C_{XYZ}), so that it is possible to three-dimensionally detect a change of the magnetic field at a position of the three sensing coils. FIG. 13 illustrates an example in which the coil sets C_{XYZ} are arranged at four corners of the panel 31 and the sensing coils C₂, C₃, C₅ to C₁₂, C₁₄, and C₁₅ are arranged at positions other than the four corners so that the rotation center axes A are in parallel with Z axis.

When the orientations of the sensing coils Cₙ are changed in this way, a plurality of sensing coils (a sensing coil group) from which output values are acquired when the determination value to be compared with the threshold is decided should be set in advance. For example, the sensing coils Cₙ, whose orientations are the same, such as, the sensing coils C₂, C₃, C₅ to C₁₂, C₁₄, and C₁₅ whose rotation center axes A are in parallel with Z axis, are set as a sensing coil group from which output values are acquired when the determination value is decided. Alternatively, the sensing coils C_{1X}, C_{4X}, C_{13X}, and C_{16X} whose rotation center axes A are in parallel with X axis or the sensing coils C_{1Y}, C_{4Y}, C_{13Y}, and C_{16Y} whose rotation center axes A are in parallel with Y axis may be set as a sensing coil group from which output values are acquired when the determination value is decided. In this case, the determination value is decided based on the output values acquired from the set sensing coil group. The determination value decision method is the same as those in the first embodiment (the determination value decision methods (1) to (4)).

In this case, the threshold should also be acquired based on output values of the sensing coils Cₙ whose orientation is the same as that of the sensing coils Cₙ used to decide the determination value. The threshold setting method is the same as those in the first embodiment (the threshold setting methods (1) or (2)) except that the sensing coil group from which output values are acquired (the orientation of each sensing coil Cₙ) is limited.

### Modified Example 3-1

Next, a modified example 3-1 of the third embodiment of the present invention will be described.

As illustrated in FIG. 13, when the sensing coils Cₙ arranged on the panel 31 are oriented to each of the XYZ directions, each of a sensing coil group whose rotation center axis A is in parallel with X axis (sensing coils C_{1X}, C_{4X}, C_{13X}, and C_{16X}), a sensing coil group whose rotation center axis A is in parallel with Y axis (sensing coils C_{1Y}, C_{4Y}, C_{13Y}, and C_{16Y}), and a sensing coil group whose rotation center axis A is in parallel with Z axis (sensing coils C₂, C₃, C₅ to C₁₂, C₁₄, and C₁₅) may be set in advance as a sensing coil group from which output values are acquired when the determination value is decided. In this case, it is possible to perform determination for each direction based on output values of each sensing coil group by deciding three determination values respectively for the XYZ directions and comparing these determination values respectively with thresholds acquired respectively for the XYZ directions.

In the case in which the determination is performed for each direction in this way, when the determination value is greater than or equal to the threshold in either of the XYZ directions, the determination unit 563 (see FIG. 1) may determine that the capsule endoscope 10 really exists in the detection target area R and proper position detection is possible.

### Modified Example 3-2

Next, a modified example 3-2 of the third embodiment of the present invention will be described.

As illustrated in FIG. 13, when the sensing coils Cₙ arranged on the panel 31 are oriented to each of the XYZ directions, the sensing coil group from which output values are acquired when the determination value is decided may include sensing coils Cₙ facing directions different from each other. As an example, in addition to the sensing coils C₂, C₃, C₅ to C₁₂, C₁₄, and C₁₅ whose rotation center axes A are in parallel with the Z direction, the four coil sets C_{XYZ} arranged at the corners of the panel 31 are set in advance as the sensing coil group from which output values are acquired.

In this case, regarding each coil set C_{XYZ}, the sum of squares of output values of three sensing coils (for example, C_{1X}, C_{1Y}, and C₁) included in one coil set C_{XYZ} is calculated and the sum of squares is used as an output value of the sensing coils when the determination value is decided. Therefore, it is possible to reliably detect the strength of the magnetic field generated by the magnetic field generating coil 141 regardless of the mutual relationship between the position and orientation of the magnetic field generating coil 141 included in the capsule endoscope 10 and the sensing coils Cₙ.

Alternatively, instead of the sum of squares of output values of three sensing coils included in one coil set C_{XYZ}, the root-mean-square is calculated, and the value of the root-mean-square may be used as an output value of the sensing coils when the determination value is decided.

### Fourth Embodiment

Next, a fourth embodiment of the present invention will be described.

In the first embodiment described above, when it is determined that proper position detection of the capsule endoscope 10 is not possible, the position detection calculation is not performed by the position detection calculation unit 564. However, the position detection calculation may be performed by the position detection calculation unit 564. In this case, the calculation unit 56 may output information indicating that the position of the capsule endoscope 10 is an error and cause the display unit 52 to display the information. Hence, it is possible for a user to perform a guidance operation of the capsule endoscope 10 after recognizing that the position of the capsule endoscope 10 displayed on the display unit 52 is an error.

Alternatively, when it is determined that proper position detection of the capsule endoscope 10 is not possible, the calculation unit 56 may stop a position display of the capsule endoscope 10 on the display unit 52. Therefore, when the position of the capsule endoscope 10 is not displayed on the display unit 52, the user can recognize that proper position detection of the capsule endoscope 10 cannot be performed.

### Fifth Embodiment

Next, a fifth embodiment of the present invention will be described.

In the first embodiment described above, when it is determined that proper position detection of the capsule endoscope 10 is not possible, the guidance control on the capsule endoscope 10 is turned off. In an opposite manner, the guidance control may be started when proper position detection of the capsule endoscope 10 becomes possible.

Specifically, the guidance system 1 starts the examination performed by using the capsule endoscope 10 in a state in which the magnetic shield 43 of the guidance magnetic field generating device 40 is closed, that is, a state in which the guidance control is not performed on the capsule endoscope 10. When the determination unit 563 determines that proper position detection of the capsule endoscope 10 is possible (see step S14 in FIG. 4), the guidance magnetic field controller 57 opens the magnetic shield 43. With this control, a guidance magnetic field is generated in a space including the detection target area R, and the guidance control on the capsule endoscope 10 can be started.

On the other hand, in the guidance system 1, the examination performed by using the capsule endoscope 10 may be started in a state in which the magnetic shield 43 of the guidance magnetic field generating device 40 is opened. In this case, when the determination unit 563 determines that proper position detection of the capsule endoscope 10 is not possible (see step S18 in FIG. 4), the guidance magnetic field controller 57 closes the magnetic shield 43. With this control, the guidance magnetic field is shielded from a space including the detection target area R, and the guidance control on the capsule endoscope 10 cannot be started.

According to some embodiments, a threshold, which is used as a reference, based on output values from a plurality of magnetic field detectors under a predetermined condition is compared with a determination value decided by using at least one of the output values from the plurality of magnetic field detectors. With this feature, it is possible to appropriately determine whether or not proper position detection of the capsule medical device is possible. Therefore, even when the capsule medical device is not located in a space of a position detection target, it is possible to prevent improper output of a position detection result of the capsule medical device.

The first to the fifth embodiments and the modified examples of the embodiments described above are merely examples for implementing the present invention, and the present invention is not limited to these embodiments and modified examples. The various inventions may be made by appropriately combining a plurality of elements disclosed in the first to the fifth embodiments and the modified examples of the embodiments. From the above description, it is obvious that the present invention can be variously modified according to specifications and the like, and further, other various embodiments are possible within the scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A position detection system comprising:
a capsule medical device having therein a magnetic field generator configured to generate a magnetic field;
a plurality of magnetic field detectors configured to detect the magnetic field generated by the magnetic field generator and output a plurality of detection signals; and
a processor comprising hardware, wherein the processor is configured to:
calculate a position of the capsule medical device by using at least one of the plurality of detection signals respectively output by the plurality of magnetic field detectors;
determine whether or not proper position detection of the capsule medical device based on the plurality of detection signals is possible by using a threshold, the threshold being a value based on at least one of output values of the plurality of detection signals respectively output by the plurality of magnetic field detectors under a predetermined condition;
decide a determination value by using at least one of the output values of the plurality of detection signals, output from at least one of the plurality of magnetic field detectors; and
determine that the proper position detection of the capsule medical device is not possible when the determination value is smaller than the threshold.

2. The position detection system according to claim 1, wherein the predetermined condition indicates that the capsule medical device is located where levels of the output values of the plurality of detection signals respectively output by the plurality of magnetic field detectors are lowest.

3. The position detection system according to claim 1, wherein the predetermined condition indicates that the capsule medical device is located at a boundary of a detection target area where the position of the capsule medical device is detectable.

4. The position detection system according to claim 1, wherein the processor is configured to decide the determination value based on all the output values from the plurality of magnetic field detectors.

5. The position detection system according to claim 1, wherein the processor is configured to decide the determination value based on output values from a predefined magnetic field detector group among the plurality of magnetic field detectors.

6. The position detection system according to claim 1, wherein the processor is configured to decide the determination value based on an output value from at least one pre-selected magnetic field detector from among the plurality of magnetic field detectors according to the output values of the plurality of detection signals under a condition that the plurality of detection signals is not affected by the magnetic field generated by the magnetic field generator.

7. The position detection system according to claim 1, wherein the processor is configured to decide, as the determination value, a maximum value of the output values of the plurality of detection signals in detecting the position of the capsule medical device.

8. The position detection system according to claim 1, wherein the processor is configured to decide the determination value by using a predetermined number of output values in descending order among the output values of the plurality of detection signals in detecting the position of the capsule medical device.

9. The position detection system according to claim 1, wherein the processor is configured to decide the determination value by using a first output value of a first detection signal output by a first magnetic field detector of the plurality of magnetic field detectors and by using second output values of second detection signals output by a predetermined number of second magnetic field detectors of the plurality of magnetic field detectors, the first output value being a maximum value of the output values of the plurality of detection signals, and the predetermined number of the second magnetic field detectors being adjacent to the first magnetic field detector.

10. The position detection system according to claim 1, wherein when the processor determines that the proper position detection of the capsule medical device is not possible, the processor is not configured to calculate the position of the capsule medical device.

11. The position detection system according to claim 1, further comprising a display configured to display information indicating that the position of the capsule medical device calculated by the processor is an error when the processor determines that the proper position detection of the capsule medical device is not possible.

12. The position detection system according to claim 1, further comprising a display configured to display the position of the capsule medical device,
wherein, when the processor determines that the proper position detection of the capsule medical device is not possible, the display is configured to stop display of the position of the capsule medical device.

13. A guidance system, comprising:
the position detection system according to claim 1 comprising the capsule medical device further having a permanent magnet;
a guidance magnetic field generator configured to generate a magnetic field to be applied to the permanent magnet; and
a guidance magnetic field controller configured to control the guidance magnetic field generator to perform guidance control for changing at least one of a position and a posture of the capsule medical device.

14. The guidance system according to claim 13, further comprising a shield device configured to shield the magnetic field generated by the guidance magnetic field generator,
wherein when the processor determines that the proper position detection of the capsule medical device is not possible, the guidance magnetic field controller is configured to cause the shield device to shield the magnetic field generated by the guidance magnetic field generator.

15. The guidance system according to claim 13, wherein the guidance magnetic field controller is configured to switch between a state in which the guidance control is possible and a state in which the guidance control is not possible, depending on whether or not the processor determines that the proper position detection of the capsule medical device is possible.
